(19) United States
(12) Patent Application Publication
(10) Pub. No.: US 2022/0044818 A1
(43) Pub. Date: Feb. 10, 2022

(54) SYSTEM AND METHOD FOR QUANTIFYING PREDICTION UNCERTAINTY

(71) Applicant: KONINKLIJKE PHILIPS N.V., Eindhoven (NL)

(72) Inventor: Yale Chang, Lincoln, MA (US)

(21) Appl. No.: 17/336,696

(22) Filed: Jun. 2, 2021

Related U.S. Application Data
(60) Provisional application No. 63/060,696, filed on Aug. 4, 2020.

(51) Int. Cl. G16H 50/30; G16H 40/20; G16H 15/00

(52) U.S. Cl. CPC G16H 50/30; G16H 15/00; G16H 40/20

(57) ABSTRACT

A method for risk analysis, comprising: (i) receiving a plurality of features about a subject; (ii) analyzing the features using risk prediction models to generate risk scores; (iii) determining, using a distillation model, mean and variance among the risk scores; (iv) generating a single risk score and a risk score confidence interval; (v) determining, based on a feature impact score for each feature, an effect of one or more missing or defective features on the generated risk score confidence interval, wherein the system identifies a missing or defective feature for reporting if that feature would narrow the generated risk score confidence interval if it were not missing or not defective; (vi) generating a report comprising the single risk score and the risk score confidence interval, and further comprising at least one or more of the identified missing or defective features; and (vii) providing the report.

### FIELD OF THE DISCLOSURE

The present disclosure is directed generally to methods and systems for quantifying the effect of missing or defective patient features on both a health risk score and the confidence interval associated with that health risk score.

### BACKGROUND

Disease risk prediction models estimate the likelihood or probability of a condition or disease occurring in the future. The models utilize information such as demographics, vital signs, clinical measures, and other subject features as input. The value or importance of an input feature depends on the condition or disease for which the likelihood or probability is being estimated. Disease risk prediction models are increasingly being used in the health care setting.

Most existing disease risk prediction models provide clinical decision support by applying a fixed threshold to the risk score generated by the model. For example, the patient may be predicted to be at risk for a condition or disease if the risk score is higher than a threshold, and may be predicted to not be at risk for the condition or disease if the risk score is below the threshold. However, a model can make overconfident predictions for patients with a risk score close to the decision boundary, and patients for whom one or more important and/or impactful subject features are missing. In each case, it would be ideal if the model could output high prediction uncertainty for the prediction, implying therefore that it is highly probable that a prediction is incorrect. However, existing approaches only focus on computing a confidence interval of the prediction.

### SUMMARY OF THE DISCLOSURE

There is a continued need for disease risk prediction models with improved risk predictions and higher confidence levels.

It would be ideal if disease risk prediction models could determine prediction uncertainty. Model uncertainty is defined as the prediction variance induced by the uncertainty of estimating model parameters. Model uncertainty can be estimated through Bayesian approaches or bootstrapping. Bayesian approaches place a prior distribution on model parameters and update the prior using observed data. The resulting posterior distribution of model parameters characterize the model uncertainty. Bayesian approaches cannot be easily applied to most prediction models, such as boosting and tree-based models, due to its requirements of prior distributions and the challenge of computing posterior distributions. On the other hand, in bootstrapping, multiple models can be trained by creating multiple datasets through sampling from the training dataset with replacement. The empirical distribution of model parameters of multiple trained models can capture model uncertainty. Compared to Bayesian approaches, bootstrapping is more flexible in that it can be combined with any prediction model.

For patients with missing or defective input features, the input uncertainty is defined as the prediction variance induced by the imperfect quality of input features. Input uncertainty can be estimated through a multiple imputation approach. Described herein is the fit of Gaussian mixture model to a dataset with missing values before applying the multiple imputation approach. Multiple imputation can output the overall value of input uncertainty, which could be due to the imperfect quality of multiple features, such as missing labs and vital signs. With this approach, the overall prediction uncertainty can be decomposed into model uncertainty and input uncertainty. Further, the methods described herein can quantify the contribution of each individual feature to the input uncertainty using the feature impact score, which is defined as the reduction of input uncertainty if the feature value is fixed across multiple imputations. The classification performance can be improved by, for example, suggesting that clinicians measure features with high feature impact scores.

Accordingly, the present disclosure is directed at inventive methods and systems for improving the disease risk analysis performed by a disease risk prediction model. Various embodiments and implementations herein are directed to a disease risk system or method that analyzes, using a plurality of different risk prediction models, a received set of features about a subject. Each of the prediction models generates a health risk score for the subject. A distillation model of the system determines an estimated mean and variance among the generated health risk scores for the subject, and utilizes that information to generate a single health risk score and a risk score confidence interval for the subject. To quantify uncertainty, the system determines, based on a predetermined feature impact score for each different type of feature, an effect of one or more missing or defective features on the generated risk score confidence interval. The system identifies a missing or defective feature for reporting if the missing or defective feature would narrow the generated risk score confidence interval if it were not missing or not defective. A report is generated comprising the single health risk score and the risk score confidence interval for the subject, and at least one or more of the missing or defective features identified for reporting. The report is then provided, which facilitates collection of updated information for the missing or defective features. The updated information may then be utilized to improve the single health risk score and the risk score confidence interval for the subject.

Generally, in one aspect, a method for risk analysis is provided. The method includes: (i) receiving a plurality of features obtained about a subject; (ii) analyzing the received plurality of features using a plurality of different risk prediction models, wherein each of the plurality of different risk prediction models generates a health risk score for the subject; (iii) determining, using a distillation model, an estimated mean and variance among the generated health risk scores for the subject; (iv) generating, from the determined estimated mean and variance, a single health risk score and a risk score confidence interval for the subject; (v) determining, based on a predetermined feature impact score for each different type of feature, an effect of one or more missing or defective features on the generated risk score confidence interval, wherein the system identifies a missing or defective feature for reporting if the missing or defective feature would narrow the generated risk score confidence interval if it were not missing or not defective; (vi) generating a report comprising the single health risk score and the risk score confidence interval for the subject, and further comprising at least one or more of the missing or defective features identified for reporting; and (vii) providing the report.

According to an embodiment, the method further includes: receiving information regarding at least one or more of the missing or defective features identified for reporting to produce an updated plurality of features about the subject; analyzing the updated plurality of features using the plurality of different risk prediction models, wherein each of the plurality of different risk prediction models generates an updated health risk score for the subject; determining, using a distillation model, an estimated mean and variance among the updated health risk scores for the subject; generating, from the determined estimated mean and variance, an updated single health risk score and an updated risk score confidence interval for the subject; generating a report comprising the updated single health risk score and the risk updated score confidence interval for the subject; and providing the report.

According to an embodiment, at least some of the received plurality of features are vital signs and test results, and wherein at least some of the plurality of features are received via an interface to an electronic health database.

According to an embodiment, the report comprises a ranking of one or more of the missing or defective features identified for reporting, the ranking based on the determined effect of each of the one or more missing or defective features on the generated risk score confidence interval.

According to an embodiment, the risk score comprises a probability of a risk, and the confidence interval comprises a range for the probability. According to an embodiment, the risk score comprises a probability of the risk being within a confidence interval range.

According to an embodiment, the report comprises at least one or more of the missing or defective features identified for reporting comprises a recommendation to obtain new data for the at least one or more of the missing or defective features. According to an embodiment, the method further comprises receiving and carrying out instructions to pause or silence the recommendation.

According to another aspect is a system configured to perform a risk analysis. The system includes: a plurality of plurality of features obtained about a subject; a plurality of risk prediction models each configured to analyze the plurality of features and further configured to generate a risk score for the subject; a distillation model configured to determine an estimated mean and variance among the generated risk scores for the subject; a processor configured to: (i) generate, from the determined estimated mean and variance, a single risk score and a risk score confidence interval for the subject; (ii) determine, based on a feature impact score for each different type of feature, an effect of one or more missing or defective features on the generated risk score confidence interval, wherein the system identifies a missing or defective feature for reporting if the missing or defective feature would narrow the generated risk score confidence interval if it were not missing or not defective; and (iii) generate a report comprising the single risk score and the risk score confidence interval for the subject, and further comprising at least one or more of the missing or defective features identified for reporting; and a user interface (640) configured to provide the report.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for reporting risk using a risk analysis system, in accordance with an embodiment.

FIG. 2 is a graph of confidence intervals, in accordance with an embodiment.

FIG. 3 is a graph of precision-recall curves, in accordance with an embodiment.

FIG. 4A is graph of a precision-recall curve for a subset of samples notified to measure new features, in accordance with an embodiment.

FIG. 4B is a graph of a precision-recall curve for a full test set, in accordance with an embodiment.

FIG. 5A is a graph of the probability of recovering a feature's value, in accordance with an embodiment.

FIG. 5B is a graph of the probability of recovering a feature's value, in accordance with an embodiment.

FIG. 6 is a schematic representation of a risk analysis system, in accordance with an embodiment.

### DETAILED DESCRIPTION OF EMBODIMENTS

The present disclosure describes various embodiments of a system and method for generating a risk report for a subject. Applicant has recognized and appreciated that it would be beneficial to provide a method and system that can improve a disease risk report by identifying missing or defective data that introduces uncertainty into a prediction. Accordingly, a disease risk system comprises a plurality of different risk prediction models each of which analyses a received set of features about a subject. The prediction models generate a plurality of health risk scores for the subject, and a distillation model of the system determines an estimated mean and variance among the generated health risk scores for the subject. The disease risk system utilizes that information to generate a single health risk score and a risk score confidence interval for the subject. To quantify uncertainty, the system determines, based on a predetermined feature impact score for each different type of feature, an effect of one or more missing or defective features on the generated risk score confidence interval. The system identifies a missing or defective feature for reporting if the missing or defective feature would narrow the generated risk score confidence interval if it were not missing or not defective. A report is generated comprising the single health risk score and the risk score confidence interval for the subject, and at least one or more of the missing or defective features identified for reporting. The report is then displayed on a user display, which facilitates collection of updated information for the missing or defective features. The updated information may then be utilized to improve the single health risk score and the risk score confidence interval for the subject.

According to an embodiment, the system comprises a confidence interval based on the distillation of bootstrapping training, and a feature impact score based on the reduction of input uncertainty. The system computes model uncertainty to construct the confidence interval, which is achieved by training multiple prediction models through bootstrapping. The mean and variance of predictions from multiple bootstrapping models can be approximated by a distillation model, and the confidence interval can be displayed together with the risk score. If the confidence interval covers the decision cut-off, indicating that the risk score could either be higher or lower than the cut-off, the system can abstain from making a prediction. The maximal abstention rate can be further enforced to determine whether the model would abstain for a given sample. The system also computes a feature impact score as the reduction of input uncertainty if the feature value was fixed across multiple imputations. By measuring features with high feature impact score, the classification performance can be effectively improved.

Although the risk analysis system is described in reference to analyzing disease risk for a subject, it should be appreciated that the risk analysis methods and systems described or otherwise envisioned here are not limited to analyzing disease risk. For example, the subject features described herein can be non-medical features about the subject, and can be related to risk assessments other than medical or health.

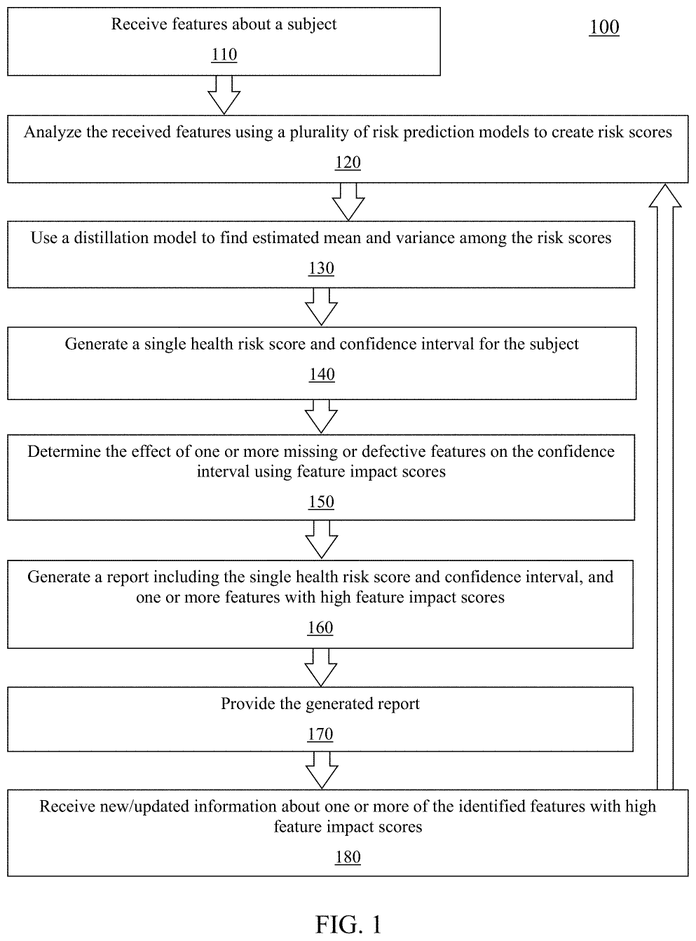

Referring to FIG. 1, in one embodiment, is a flowchart of a method 100 for generating, using a risk analysis system, an improved risk estimate. The risk analysis system can be any of the risk analysis systems described or otherwise envisioned herein.

At step 110 of the method, the disease risk analysis system receives a plurality of features for a subject. The subject can be a patient or any other individual for which a risk assessment will be performed. For example, the subject may be a patient in a healthcare setting such as a healthcare provider's office, an emergency setting, an in-patient facility, an out-patient facility, and/or any other setting where a risk assessment may be performed.

According to an embodiment, a feature can be anything relevant to a subject and/or to a disease risk assessment. For example, a feature can comprise medically relevant information about a subjection, including but not limited to demographics, physiological measurements such as vital data, injury information, physical observations, clinical test results, and/or diagnosis, among many other types of medical information. As an example, the medical information can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Many other types, categories, or variations of features are possible.

A feature can be obtained in a wide variety of different ways. For example, a feature may be manually input into the disease risk analysis system via a user interface. A feature may be retrieved from an electronic health database in response to a query from the disease risk analysis system, or the electronic health database may feed the feature to the disease risk analysis system in response to direction to do so. Thus, the disease risk analysis system can be in wired and/or wireless communication with an electronic health database, or the disease risk analysis system may comprise or be a component of a system including an electronic health database. A feature may be provided to or otherwise obtained by the disease risk analysis system.

At step 120 of the method, the disease risk analysis system analyzes the received plurality features using a plurality of different risk prediction models. Each of the plurality of different risk prediction models generates a health risk score for the subject. A risk prediction model can be any model trained or otherwise configured, programmed, or designed to generate a risk score based on one or more input features. As described or otherwise envisioned herein, one or more of the risk prediction models can be trained using training datasets that may be specific to the healthcare setting or a more generic training dataset. Pursuant to a bootstrapping approach, multiple models can be trained by creating multiple datasets through sampling from the training dataset with replacement. The empirical distribution of model parameters of multiple trained models can therefore capture model uncertainty as described below. According to an embodiment, the each of the plurality of different risk prediction models may perform a risk assessment once or multiple times.

At step 130 of the method, the disease risk analysis system determines an estimated mean and variance among the generated health risk scores for the subject using a distillation model. The distillation model may be any model or process capable of distilling the output of the plurality of different risk prediction models.

At step 140 of the method, the disease risk analysis system generates, from the determined estimated mean and variance, a single health risk score and a risk score confidence interval for the subject. This single health risk score and the risk score confidence interval comprises model uncertainty which can be assessed and ameliorated as discussed or otherwise described herein.

Definition of Confidence Interval

In binary classification (with class label unstable (1) and stable (0)), one can use r(x)=p(y=1|x) to represent the probability that the input sample x ∈ [symbol] ^{D} is predicted in the unstable (disease) class. r(x) is often created by applying the sigmoid function sigmoid(⋅) to its logit score f(x) as follows:

$$r ( x ) = \frac{1}{1 + {e}^{- f ( x )}} \qquad ( Eq . 1 )$$

As just one example, the model used to predict hemodynamic instability is based on a known abstain adaboost classifier, where f(x) is modelled as the weighted average of 200 decision stumps.

According to an embodiment, the disease risk analysis system and method is interested in learning the predictive distribution of f(x). Suppose the predictive distribution of f(x) can be approximated by a Gaussian distribution:

f(x)˜ [symbol] (μ(x), σ²(x)) (Eq. 2)

Then the 95% confidence interval of f(x) can be derived from the empirical rule of Gaussian distribution as:

CI_{95%}(f(x))=[μ(x)−2σ(x), μ(x)+2σ(x)] (Eq. 3)

The 95% confidence interval of r(x) can be derived by applying the sigmoid function to the lower bound and upper bound as:

CI_{95%}(r(x))=[sigmoid(μ(x)−2σ(x)), sigmoid(μ(x)+2σ(x))] (Eq. 4)

Definition of Model Uncertainty

The model uncertainty and input uncertainty are two sources contributing to the variance of f(x).

Models trained using patient cohorts collected from different hospitals would be different. Models trained using different subsets of the same hospital would also be different. During training, although there may be access to patients collected from multiple hospitals, the system may still need to consider the potential model variation using different patient cohorts, which can be simulated through bootstrapping.

According to an embodiment, model uncertainty is defined as the prediction variance due to the variation of the trained model f_{θ}(⋅) given potential different training dataset, where θ represents the model parameters. The distribution of model parameters can capture the model variation induced by the variation of training datasets. Model uncertainty is useful because it would be higher for patients' data closer to the decision boundary.

At step 150 of the method, the disease risk analysis system determines an effect of one or more missing or defective features on the generated risk score confidence interval using a feature impact score for the feature. The system can identify a missing or defective feature for reporting if the missing or defective feature would narrow the generated risk score confidence interval if it were not missing or not defective.

Definition of Input Uncertainty

Input uncertainty can be defined as the prediction variance due to the imperfect quality of the input data, including missing features, unreliable feature values due to their old ages, and measurement noise.

The method can denote the observed feature vector as x and the processed feature vector as z, which is derived by applying pre-processing steps on x, including 1) imputation to fill in missing values, which can be used to quantify the influence of missing values on input uncertainty; and 2) renewing old temporal measurements, which can be used to quantify the bias of old-aged measurements on input uncertainty. These pre-processing steps can be captured by p(z|x), the conditional distribution of processed input z given raw input x.

Input uncertainty is useful because it would be higher for patients whose input data quality is low. For example, if hemoglobin is missing for a patient the resulting high input uncertainty can be used to make the classifier abstain from making predictions for hemoglobin.

Definition of Feature Impact Score

The contribution of each feature to the input uncertainty can be further quantified by computing the feature impact score (FIS) for each feature, which is defined as the reduction of input uncertainty if the feature's quality is ideal. The FIS is useful because its values would be high for features that: 1) are predictive of the outcome variable; and 2) have low quality, such as being missing. Therefore, features can be ranked by their FIS values and the system can recommend that clinicians improve the quality of poor or missing features having high FIS values. Because these features are predictive of the outcome variable, improving their qualities—such as taking new measurements—will also improve the classification performance.

For example, consider the patient with a few lab values missing. The FIS of the missing lab values can be computed by the reduction of input uncertainty if these lab values were measured, which can be simulated through multiple imputation. In the missing feature example, it is shows that FIS can be interpreted as patient-level feature importance score for missing features. After computing the FIS of these missing features, the system suggests to clinicians that the variables having the highest FIS values be measured. In this way, the input uncertainty can be actively reduced and the classification performance can be improved.

Decomposition of Prediction Variance

The prediction variance can be decomposed as the summation of model uncertainty and input uncertainty. The decomposition can provide formal definitions of these two kinds of uncertainties, and can illustrate how to estimate them given observed data.

According to an embodiment, the prediction mean μ(x) can be evaluated as:

μ(x)= [symbol] _{p(z|x)} [symbol] _{p(θ)}[f_{θ}(z)] (Eq. 5)

The prediction variance σ²(x) can be decomposed into model uncertainty and input uncertainty:

σ²(x)=σ²_{model}+σ²ᵢₙₚᵤₜ (Eq. 6)

where the model uncertainty can be evaluated as:

σ²_{model}= [symbol] _{p(z|x)}[Var_{p(θ)}[f_{θ}(z)]] (Eq. 7)

According to an embodiment, the input uncertainty can be evaluated as:

σ²ᵢₙₚᵤₜ=Var_{p(z|x)}[ [symbol] _{p(θ)}[f_{θ}(z)]]

Confidence Interval Based on Model Uncertainty

According to an embodiment, p(θ), the distribution over model parameters, can be simulated by training M models using M bootstrapping datasets, which are created by randomly sampling from the training set with replacement. Denote the parameters of M trained models as {θ⁽¹⁾, . . . , θ^{(M)}}, p(θ) can be represented as the empirical distribution:

$$p ( θ ) = \frac{1}{M} {\sum }_{m = 1}^{M} δ ( θ - {θ}^{( m )} ) \qquad ( Eq . 8 )$$

p(z|x), the distribution of processed input given the raw input, can be simulated by applying multiple imputation. Denote the S imputed inputs are represented as {z⁽¹⁾, . . . , z^{(S)}}.

However, this involves running M trained models at the test time, which could be computationally expensive at the test time if M is large. Therefore, we proposed to train two distillation models to approximate the estimated mean and variance across multiple models as follows

$$\begin{matrix}{μ}_{d i s t i l l} ( {z}^{( s )} ) \approx \frac{1}{M} {\sum }_{m = 1}^{M} {f}_{θ}^{( m )} ( {z}^{( s )} ) & ( Eq . 9 ) \\ {σ}_{d i still}^{2} ( {z}^{( s )} ) \approx \frac{1}{( M - 1 )} {\sum }_{m = 1}^{M} {( {f}_{θ}^{( m )} ( {z}^{( s )} ) - \hat{μ} ( {z}^{( s )} ) )}^{2} & ( Eq . 10 ) \\ where \hat{μ} ( {z}^{( s )} ) = \frac{1}{M} {\sum }_{m = 1}^{M} {f}_{θ}^{( m )} ( {z}^{( s )} ) & ( Eq . 11 )\end{matrix}$$

According to an embodiment, the model uncertainty can be estimated as:

$${\hat{σ}}_{model}^{2} \approx \frac{1}{s} {\sum }_{s = 1}^{S} {σ}_{d i s t i l l}^{2} ( {z}^{( s )} ) \qquad ( Eq . 12 )$$

According to an embodiment, the prediction mean can be estimate as

$$\hat{μ} ( x ) = \frac{1}{S} {\sum }_{s = 1}^{S} {μ}_{d i still} ( {z}^{( s )} ) \qquad ( Eq . 13 )$$

According to an embodiment, the 95% confidence interval based on the input uncertainty can be derived from the estimated \hat{μ}(x) and \hat{θ}²_{model}:

CI_{95%}(r(x))≈[\hat{μ}(x)−2\hat{σ}_{model}, \hat{μ}(x)+2\hat{σ}_{model}] (Eq. 14)

Feature Impact Score Based on Input Uncertainty

According to an embodiment, the input uncertainty can be estimated as follows:

$${\hat{σ}}_{i n p u t}^{2} \approx \frac{1}{S - 1} {\sum }_{s = 1}^{S} {( {μ}_{d i still} ( {z}^{( s )} ) - \hat{μ} ( x ) )}^{2} \qquad ( Eq . 15 )$$

The feature impact score of the d-th feature F_{d} can be computed as the reduction of the prediction variance induced by the input uncertainty when that feature's value is fixed as the population mean across multiple imputations. To normalize the feature impact score, the reduction of the prediction interval width can be considered, where the prediction interval is defined by the prediction mean and the input uncertainty.

FIS(F_{d})=w−w_{-d} (Eq. 16)

w=sigmoid(\hat{μ}+2\hat{σ}ᵢₙₚᵤₜ)−sigmoid(\hat{μ}−2\hat{σ}ᵢₙₚᵤₜ) (Eq. 17)

w_{-d}=sigmoid(\hat{μ}+2\hat{σ}_{input|z}_{^{(s)}}_{=const})−sigmoid(\hat{μ}−2\hat{σ}_{input|z}_{^{(s)}}_{=const}) (Eq. 18)

At step 160 of the method, the disease risk analysis system generates a report that includes the single health risk score and the risk score confidence interval for the subject. The report also includes at least one or more of the missing or defective features identified for reporting. The report can be generated by the disease risk analysis system using any method for gathering, processing, and/or collating the reported information.

According to an embodiment, the report includes a ranking of one or more of the missing or defective features identified for reporting, where the ranking is based at least in part on the determined effect of each of the one or more missing or defective features on the generated risk score confidence interval. The report may comprise any other information received or generated by the risk analysis system.

At step 170 of the method, the report is provided via a user interface or other communication method. The user interface can be any device or system that allows information to be conveyed and/or received, and may include a display, a mouse, and/or a keyboard for receiving user commands. The user interface may be located with one or more other components of the system, or may located remote from the system and in communication via a wired and/or wireless communications network. For example, the report may be displayed on a screen, printed, texted, emailed, displayed via a wearable device, or provided using any other method for communicating information.

According to an embodiment, the report is provided to a clinician, which enables the clinician to evaluate the risk prediction in light of information about missing or defective features. This improves the clinician's confidence in the risk prediction. The clinician is then able to evaluate whether obtaining updated information for missing or defective features would improve the risk prediction, and can thus decide that new information regarding the missing or defective feature shall be obtained and provided to the risk prediction system.

According to an embodiment, the clinician can provide instructions to the risk analysis system to pause the recommendation, or to silence the recommendation. The pause or silence may be temporary or permanent, depending on the wishes of the clinician and/or the settings of the system. For example, the clinician may determine that the recommended one or more missing or defective features are not necessary, cannot be obtained, or are being obtained but require additional time such as for the results of a test. Other reasons for pausing or silencing a recommendation exist. Accordingly, the risk analysis system is configured to receive instructions to pause or silence a recommendation, and thus configured to pause or silence a recommendation permanently or for a predetermined or selected amount of time.

Thus, at step 180 of the method, the disease risk analysis system receives one or more features about the patient, where the feature is one of the reported missing or defective features having an impact on the previous risk assessment. For example, a clinician may gather new vital signs or laboratory testing as recommended by the report provided in step 160, and that new vital information or test results can be provided to the disease risk analysis system.

According to an embodiment, the disease risk analysis system is then prompted to perform a new risk assessment with the updated information. Accordingly, the system can analyze the updated plurality of features using the plurality of different risk prediction models to generate updated health risk scores for the subject. The distillation model determines estimated mean and variance among the updated health risk scores for the subject, and an updated single health risk score and an updated risk score confidence interval for the subject. The system can then generate and provide a report comprising the updated single health risk score and the risk updated score confidence interval for the subject.

### EXAMPLE

Discussed below is an example of an application of one embodiment of the risk analysis system and method described or otherwise envisioned herein. It will be understood that this is only one embodiment and that nothing in this example limits the scope of the claims or application.

According to an embodiment, the risk analysis system and method was applied to a hemodynamic instability patient cohort. The objective was to predict whether a patient would become hemodynamically instable within an hour, which is a binary classification task. The dataset was split into four datasets: 1) training set; 2) distillation set; 3) calibration set; and 4) test set. The distillation set was used to train distillation models to approximate the mean and variance of multiple models trained from multiple bootstrapping datasets. The calibration set was used to calibrate the risk score such that it could match the empirical probability of instability. The number of samples and prevalence of unstable patients in each set is summarized in Table 1.

TABLE 1. Size and Prevalence of Training Set, Distillation Set, Calibration Set and Test Set

TABLE 1

| Set | Training | Distillation | Calibration | Test |
|---|---|---|---|---|
| # Patients | 173,095 | 17,289 | 17,275 | 8657 |
| Prevalence | 15.2% | 15.2% | 15.1% | 14.7% |

According to an embodiment, the abstain adaboost classifier was utilized to classify the patient into the stable/unstable class, although many other classifiers are possible. Platt scaling was also applied to calibrate the risk score output by the boosting model.

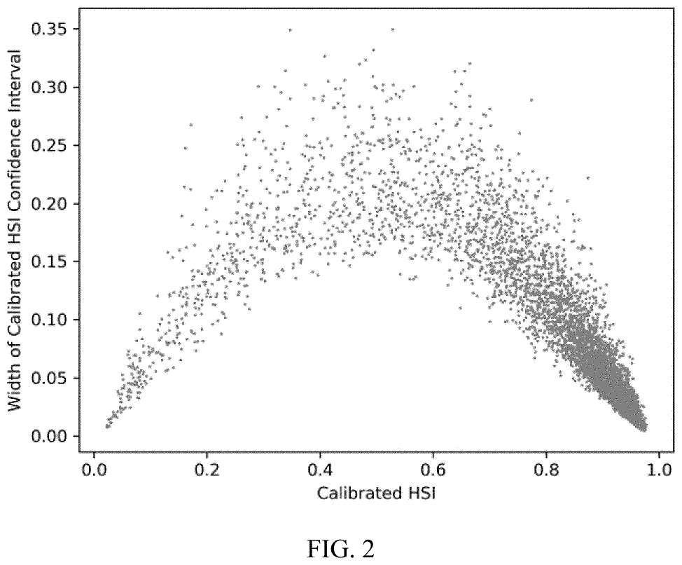

To investigate the property of the confidence interval, width of the 95% confidence interval of 1−r(x) against 1−r(x) is plotted in FIG. 2. As the graph shows, as patients move closer to the decision boundary, the width of the confidence interval increases. This indicates that patients closer to the decision boundary have higher model uncertainty.

For a given threshold, the classifier can abstain from making predictions for samples whose confidence intervals cover the decision cut-off. For example, one can set an upper bound on the abstention rate, denoted as maxAbstentionRate. For patients whose confidence intervals cover the cut-off, these patients can be ranked by the distance between the cutoff and the lower/upper bound of the confidence interval as follows: (1) if the patient's risk of instability is greater than the cut-off, then the distance between the cut-off and the lower bound of the confidence interval is computed; and (2) otherwise, the upper bound is used to compute the distance.

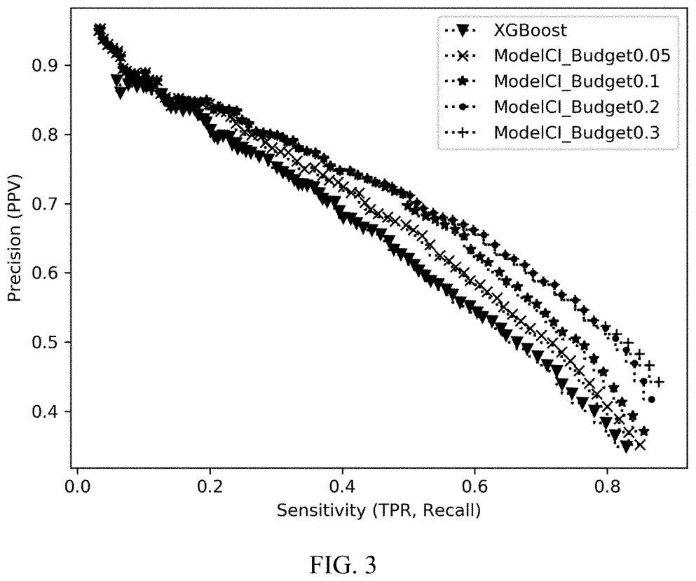

After ranking these patients, the classifier chose to abstain from making predictions for the top-ranking patients falling within the set indicated by maxAbstentionRate. The maxAbstentionRate was varied from 0.05 to 0.2. To test how this strategy (AbstainAdaBoost-CI) affects the precision (PPV), sensitivity (recall) of the unstable class, the cut-off threshold was varied and the precision-recall curves were plotted, as shown in FIG. 3. Thus, the figure shows the precision recall curve of the abstain adaboost classifier on the test set, the precision recall curves of the abstain adaboost classifier with confidence interval (no prediction is output if the confidence interval covers the cut-off), and the precision recall curves corresponding to setting the upper bound of the abstention rate maxAbstentionRate as 0.05, 0.1, 0.2. The usage of confidence interval can effectively improve the classification performance measured by the precision-recall curve.

For the same sensitivity value, the precision of AbstainAdaBoost-CI is significantly higher than AbstainAdaBoost, the baseline classifier without utilizing the confidence interval. For example, setting maxAbstentionRate=0 .1, when sensitivity is equal to 0.61 (the corresponding cut-off is 0.233), the precision of AbstainAdaBoost is 0.484. In contrast, the precision of AbstainAdaBoost-CI is 0.567. Furthermore, the margin of benefits becomes very small as maxAbstentionRate becomes greater than 0.2.

Since the feature impact score (FIS) would be high for missing features that: 1) are predictive of the outcome; and 2) cannot be estimated accurately from measured features, it is hypothesized that after actively measuring the values of (missing) features having high FIS values, the classifier is more likely to make correct prediction for the patient. Therefore, FIS can be interpreted as patient-level feature importance score for missing values.

To test this hypothesis, an experiment as described as performed. (1) For each test patient, remove 50% measured variables, and the resulting data matrix is denoted as RemovedHalf. Denote the set of removed variables as R, which is constrained to exclude heartRate and three ventilation-related variables: FiO2, Mean_Airway_Pressure, Peak_Insp_Pressure. (2) For each test patient, compute FIS for each feature in R. Recover the measured values of features with FIS values greater than a given threshold T, where the resulting data matrix is denoted as FIS. (3) Recover the measured values of the same number of features randomly selected from R, the resulting data matrix is denoted as Random. (4) The data matrix of the original test set is denoted as Original.

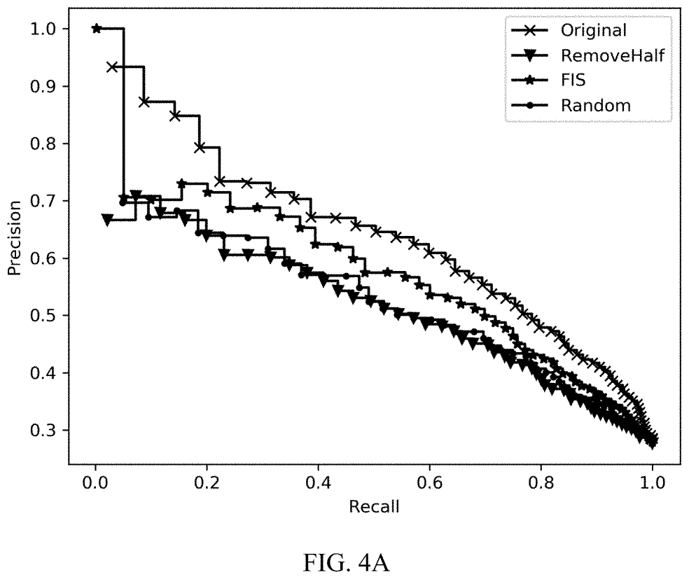

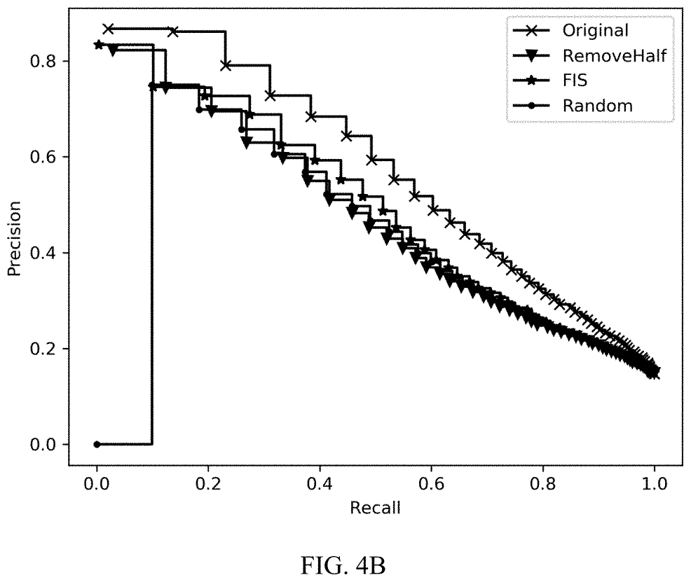

The results of the experiment are shown in FIGS. 4A and 4B. Setting the threshold $T=0.1$, FIG. 4A shows the precision-recall curve on the subset of samples who are notified to measure new features, and FIG. 4B shows the precision-recall curve on the full test set. The AUC values of different input datasets when T=0.1 is shown in Table 2.

TABLE 2
AUC Values

| Dataset | AUC (full test set) | AUC (20% test set) |
|---|---|---|
| RemoveHalf | 0.786 | 0.745 |
| Random | 0.790 | 0.754 |
| FIS | 0.798 | 0.785 |
| Original | 0.841 | 0.831 |

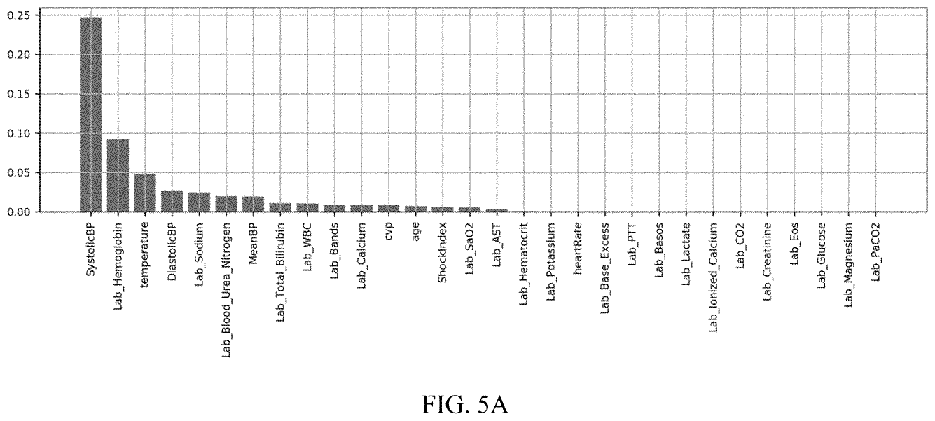

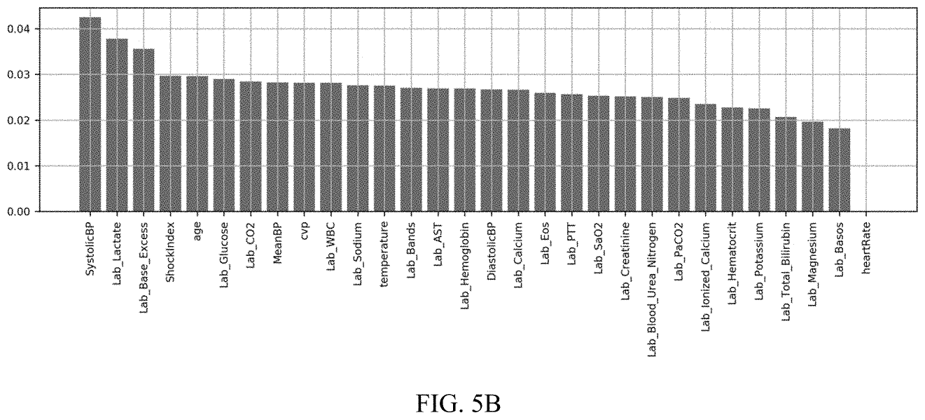

Referring to FIGS. 5A and 5B, where the threshold is set to T=0.1, FIG. 5A shows the probability of recovering a feature's value using FIS given its value was randomly removed, and FIG. 5B shows the results of the Random strategy.

The threshold T is set to be 0.1, which achieves a reasonable trade-off between the cost of measuring new features and the improvement of the classification performance. When setting T=0.1, among 8657 test samples, 20% samples were notified to measure new measures. Among these 20% test samples, each sample was notified to measure 1.2 features on average. The classification performance was evaluated on both the 20% subset and the full test set.

Based on the results, the following observations were made: (1) FIS outperforms Random in terms both precision-recall curve and AUC values. This is because the variables identified by random sampling are not necessarily predictive of the outcome. In contrast, FIS can identify variables that are missing and predictive. (2) Random would recover feature values with almost equal probability. In contrast, FIS would prefer recover feature values of SystolicBP, Lab_Hemoglobin, temperature, DiastolicBP and Lab_Sodium. Therefore, FIS can be interpreted as patient-level feature importance score for missing features.

Thus, the experiment computed 95% confidence interval of predicted risk based on model uncertainty. The model uncertainty can be used to make the classifier abstain from making predictions for patients whose confidence intervals cover the cutoff value. The resulting classifier has better classification performance measured by the precision recall curve. The input uncertainty can be used to measure the input data quality. In particular, the feature impact score was defined to measure the contribution of each feature's missingness to the input uncertainty. Actively measuring variables with highest FIS values would also improve the classification performance.

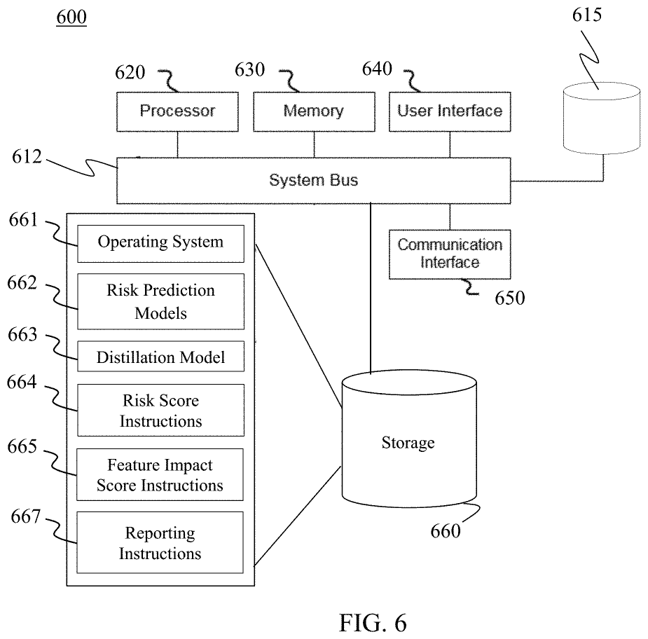

Referring to FIG. 6, in one embodiment, is a schematic representation of a risk assessment system 600. System 600 may be any of the systems described or otherwise envisioned herein, and may comprise any of the components described or otherwise envisioned herein.

According to an embodiment, system 600 comprises one or more of a processor 620, memory 630, user interface 640, communications interface 650, and storage 660, interconnected via one or more system buses 612. It will be understood that FIG. 6 constitutes, in some respects, an abstraction and that the actual organization of the components of the system 600 may be different and more complex than illustrated.

According to an embodiment, system 600 comprises a processor 620 capable of executing instructions stored in memory 630 or storage 660 or otherwise processing data to, for example, perform one or more steps of the method. Processor 620 may be formed of one or multiple modules. Processor 620 may take any suitable form, including but not limited to a microprocessor, microcontroller, multiple microcontrollers, circuitry, field programmable gate array (FPGA), application-specific integrated circuit (ASIC), a single processor, or plural processors.

Memory 630 can take any suitable form, including a non-volatile memory and/or RAM. The memory 630 may include various memories such as, for example L1, L2, or L3 cache or system memory. As such, the memory 630 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 600. It will be apparent that, in embodiments where the processor implements one or more of the functions described herein in hardware, the software described as corresponding to such functionality in other embodiments may be omitted.

User interface 640 may include one or more devices for enabling communication with a user. The user interface can be any device or system that allows information to be conveyed and/or received, and may include a display, a mouse, and/or a keyboard for receiving user commands. In some embodiments, user interface 640 may include a command line interface or graphical user interface that may be presented to a remote terminal via communication interface 650. The user interface may be located with one or more other components of the system, or may located remote from the system and in communication via a wired and/or wireless communications network.

Communication interface 650 may include one or more devices for enabling communication with other hardware devices. For example, communication interface 650 may include a network interface card (NIC) configured to communicate according to the Ethernet protocol. Additionally, communication interface 650 may implement a TCP/IP stack for communication according to the TCP/IP protocols. Various alternative or additional hardware or configurations for communication interface 650 will be apparent.

Storage 660 may include one or more machine-readable storage media such as read-only memory (ROM), random-access memory (RAM), magnetic disk storage media, optical storage media, flash-memory devices, or similar storage media. In various embodiments, storage 660 may store instructions for execution by processor 620 or data upon which processor 620 may operate. For example, storage 660 may store an operating system 661 for controlling various operations of system 600.

It will be apparent that various information described as stored in storage 660 may be additionally or alternatively stored in memory 630. In this respect, memory 630 may also be considered to constitute a storage device and storage 660 may be considered a memory. Various other arrangements will be apparent. Further, memory 630 and storage 660 may both be considered to be non-transitory machine-readable media. As used herein, the term non-transitory will be understood to exclude transitory signals but to include all forms of storage, including both volatile and non-volatile memories.

While system 600 is shown as including one of each described component, the various components may be duplicated in various embodiments. For example, processor 620 may include multiple microprocessors that are configured to independently execute the methods described herein or are configured to perform steps or subroutines of the methods described herein such that the multiple processors cooperate to achieve the functionality described herein. Further, where one or more components of system 600 is implemented in a cloud computing system, the various hardware components may belong to separate physical systems. For example, processor 620 may include a first processor in a first server and a second processor in a second server. Many other variations and configurations are possible.

According to an embodiment, system 600 may comprise or be in remote or local communication with a database or data source 615. Database 615 may be a single database or data source or multiple. Database 615 may comprise the input data which may be used to train the system, as described and/or envisioned herein.

According to an embodiment, storage 660 of system 600 may store one or more algorithms and/or instructions to carry out one or more functions or steps of the methods described or otherwise envisioned herein. For example, processor 620 may comprise one or more of risk prediction models 662, a distillation model 663, risk score instructions 664, feature impact score instructions 665, and reporting instructions 667.

According to an embodiment, a plurality of risk prediction models 662 analyze a set of received features about a subject, and each of the plurality of different risk prediction models generates a health risk score for the subject. A risk prediction model can be any model trained or otherwise configured, programmed, or designed to generate a risk score based on one or more input features. According to an embodiment, the each of the plurality of different risk prediction models may perform a risk assessment once or multiple times.

According to an embodiment, a distillation model 663 determines an estimated mean and variance among the generated risk scores for the subject. The distillation model may be any model or process capable of distilling the output of the plurality of different risk prediction models.

According to an embodiment, risk score instructions 664 direct the system to generate, from the determined estimated mean and variance, a single health risk score and a risk score confidence interval for the subject. This single health risk score and the risk score confidence interval comprises model uncertainty which can be assessed and ameliorated as discussed or otherwise described herein.

According to an embodiment, feature impact score instructions 665 direct the system to determine an effect of one or more missing or defective features on the generated risk score confidence interval using a feature impact score for the feature, as described or otherwise envisioned herein. According to an embodiment, the system can identify a missing or defective feature for reporting if the missing or defective feature would narrow the generated risk score confidence interval if it were not missing or not defective.

According to an embodiment, reporting instructions 667 direct the system to generate and provide the risk analysis report. The risk analysis report comprises the single health risk score and the risk score confidence interval for the subject, along with an identification of one or more of the missing or defective features identified for reporting by the feature impact score instructions 665. According to an embodiment, the report includes a ranking of one or more of the missing or defective features identified for reporting, where the ranking is based at least in part on the determined effect of each of the one or more missing or defective features on the generated risk score confidence interval. The report may comprise any other information received or generated by the risk analysis system. The reporting instructions 265 also direct the system to display the report on a display of the system or provide the report via any other communication mechanism or method. For example, the report may be communicated by wired and/or wireless communication to another device. For example, the system may communicate the report to a mobile phone, computer, laptop, wearable device, and/or any other device configured to allow display and/or other communication of the report.

According to an embodiment, the risk analysis system is configured to process many thousands or millions of datapoints during analysis of features by the plurality of different risk prediction models, the distillation of the risk scores into an estimated mean and variance and then generation of a single health risk score and a risk score confidence interval, and determining the feature impact score of various features on the confidence interval, among other calculations and analyses. This can require millions or billions of calculations to generate a single report comprising the single health risk score and the risk score confidence interval for the subject, along with an identification of one or more of the missing or defective features identified for reporting by the feature impact score instructions. Generating this information and providing the report comprises a process with a volume of calculation and analysis that a human brain cannot accomplish in a lifetime, or multiple lifetimes.

By providing such an improved risk analysis, the risk analysis methods and systems described or otherwise envisioned herein improve the ability of clinicians or other decisionmakers to assess risk and improve outcomes. It also increases the decisionmaker's confidence in the underlying system. As just one example, by providing a system that can identify missing or defective features that, if provided, would improve a risk assessment, the system informs decisionmakers how to easily improve risk assessment. This recommendation or call to action improves the care of the subject by providing a clearer picture of risk and a better prediction of the future. Improved risk analysis, such as that performed by the novel systems and methods described or otherwise envisioned herein, saves lives and saves millions of dollars a year in healthcare costs, when applied in the healthcare setting.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for generating a confidence interval for a risk score using a risk analysis system, comprising:
receiving a plurality of features obtained about a subject;
analyzing the received plurality of features using a plurality of different risk prediction models, wherein each of the plurality of different risk prediction models generates a health risk score for the subject;
determining, using a distillation model, an estimated mean and variance among the generated health risk scores for the subject;
generating, from the determined estimated mean and variance, a single health risk score and a risk score confidence interval for the subject;
determining, based on a predetermined feature impact score for each different type of feature, an effect of one or more missing or defective features on the generated risk score confidence interval, wherein the system identifies a missing or defective feature for reporting if the missing or defective feature would narrow the generated risk score confidence interval if it were not missing or not defective;
generating a report comprising the single health risk score and the risk score confidence interval for the subject, and further comprising at least one or more of the missing or defective features identified for reporting; and
providing the report.

2. The method of claim 1, further comprising the steps of:
receiving information regarding at least one or more of the missing or defective features identified for reporting to produce an updated plurality of features about the subject;
analyzing the updated plurality of features using the plurality of different risk prediction models, wherein each of the plurality of different risk prediction models generates an updated health risk score for the subject;
determining, using a distillation model, an estimated mean and variance among the updated health risk scores for the subject;
generating, from the determined estimated mean and variance, an updated single health risk score and an updated risk score confidence interval for the subject;
generating a report comprising the updated single health risk score and the risk updated score confidence interval for the subject; and
providing the report.

3. The method of claim 1, wherein at least some of the received plurality of features are vital signs and test results, and wherein at least some of the plurality of features are received via an interface to an electronic health database.

4. The method of claim 1, wherein the report comprises a ranking of one or more of the missing or defective features identified for reporting, the ranking based on the determined effect of each of the one or more missing or defective features on the generated risk score confidence interval.

5. The method of claim 1, wherein the risk score comprises a probability of a risk, and the confidence interval comprises a range for the probability.

6. The method of claim 1, wherein the risk score comprises a probability of the risk being within a confidence interval range.

7. The method of claim 1, wherein the report comprises at least one or more of the missing or defective features identified for reporting comprises a recommendation to obtain new data for the at least one or more of the missing or defective features.

8. The method of claim 7, further comprising the step of receiving and carrying out instructions to pause or silence the recommendation.

9. A system configured to generate a confidence interval for a risk score using a risk analysis system, comprising:
a plurality of plurality of features obtained about a subject;
a plurality of risk prediction models each configured to analyze the plurality of features and further configured to generate a risk score for the subject;
a distillation model configured to determine an estimated mean and variance among the generated risk scores for the subject;
a processor configured to: (i) generate, from the determined estimated mean and variance, a single risk score and a risk score confidence interval for the subject; (ii) determine, based on a feature impact score for each different type of feature, an effect of one or more missing or defective features on the generated risk score confidence interval, wherein the system identifies a missing or defective feature for reporting if the missing or defective feature would narrow the generated risk score confidence interval if it were not missing or not defective; and (iii) generate a report comprising the single risk score and the risk score confidence interval for the subject, and further comprising at least one or more of the missing or defective features identified for reporting; and
a user interface configured to provide the report.

10. The system of claim 9, wherein the processor is further configured to:
receive information regarding at least one or more of the missing or defective features identified for reporting to produce an updated plurality of features about the subject; and
perform a new risk analysis with the updated plurality of features.

11. The system of claim 9, wherein at least some of the received plurality of features are vital signs and test results, and wherein at least some of the plurality of features are received via an interface to an electronic health database.

12. The system of claim 9, wherein the report comprises a ranking of one or more of the missing or defective features identified for reporting, the ranking based on the determined effect of each of the one or more missing or defective features on the generated risk score confidence interval.

13. The system of claim 9, wherein the risk score comprises a probability of a risk, and the confidence interval comprises a range for the probability.

14. The system of claim 9, wherein the risk score comprises a probability of the risk being within a confidence interval range.

15. The system of claim 9, wherein the report comprises at least one or more of the missing or defective features identified for reporting comprises a recommendation to obtain new data for the at least one or more of the missing or defective features.
